(19) United States
(12) Patent Application Publication
(10) Pub. No.: US 2025/0248855 A1
(43) Pub. Date: Aug. 7, 2025

(54) EASILY REPLACEABLE ADHESIVE BANDAGE FOR ADVANCED WOUND HEALING AND METHOD

(71) Applicant: JOHNSON & JOHNSON CONSUMER INC., Skillman, NJ (US)

(72) Inventors: Cristina CASTRO, Skillman, NJ (US); Jenny DU-SORIANO, New Hope, PA (US); Latrisha PETERSEN, Raritan, NJ (US); Vivian PIERRI, São Paulo (BR)

(21) Appl. No.: 18/429,879

(22) Filed: Feb. 1, 2024

(51) Int. Cl. A61F 13/0206; A61F 13/00; A61F 13/0246; A61L 15/58

(52) U.S. Cl. CPC A61F 13/0209; A61F 13/00085; A61F 13/0253; A61L 15/58; A61F 2013/00089

(57) ABSTRACT

An adhesive bandage having a backing material and a hydrocolloid pad disposed thereon effectively removes wound exudate while enabling facile removal. The bandage includes a backing material comprising a sheet having an adhesive layer disposed thereon having a first color. The hydrocolloid pad has an absorbency (24 hr) of between about 2 and about 6 g/g, and includes a hydrocolloid layer forming a first surface thereof and a polymeric film layer having a second color associated therewith different than the first color of the backing material forming a second surface thereof. The hydrocolloid pad is disposed in facing relation and adhered to the adhesive layer of backing material. The first surface having a dry peel strength of between about 0 and about 700 gf, a wet peel strength of between about 0 and 150 gf.

### FIELD OF THE INVENTION

The present invention relates generally to novel adhesive bandages and methods of use for advanced wound healing.

### BACKGROUND OF THE INVENTION

The treatment of minor wounds, cuts and abrasions on the skin has included the use of various types of wound dressings and bandages. Among these, adhesive bandages that can be applied by an individual as a self-care treatment are preferred. Typically, adhesive bandages are comprised of a backing material with a pad affixed to the backing material. The intended skin-facing ends of the backing material on either side of or surrounding the pad typically have a biocompatible adhesive applied. The pad contacts the wound and protects the wound from further incidental contact and associated discomfort. The pad of the adhesive bandage may be absorbent and comprise cotton or other natural fibers, polymeric fibers, hydrogel or hydrocolloid materials, or the like. Wound exudate may be absorbed to different degrees depending on the nature of the pad.

Nuutila & Eriksson in “Moist Wound Healing with Commonly Available Dressings” Adv Wound Care (New Rochelle) 2021 December;10 (12): 685-698 (doi: 10.1089/wound.2020.1232) state that hydrocolloids maintain a moist environment conducive to wound healing by forming a gel with the wound fluid or exudate. Wounds heal faster in a moist environment. The moist wound environment facilitates autolytic debridement, reduces pain, reduces scarring, activates collagen synthesis, facilitates and promotes keratinocyte migration over the wound surface, and supports the presence and function of nutrients, growth factors, and other soluble mediators in the wound microenvironment. It has also been shown that immediate wound treatment with moist dressings decreases tissue loss and mitigates wound progression, especially in burns. Many studies have demonstrated that healing in a moist environment results in less inflammation than in a dry environment and thus also improves the quality of healing (less scarring). Common hydrocolloid dressings include DuoDERM®, CombiDERM™, and GranuGel™. The wear time of hydrocolloid dressings is usually several days. As an example, the manufacturer of DuoDERM hydrocolloid dressing instructs that it can be worn continuously for up to 7 days unless it is uncomfortable, there is leakage, or there are clinical signs of infection. Further, the DuoDERM use instructions indicate that frequent dressing changes on wounds with damaged or delicate skin surrounding the wound is not recommended.

The force required to remove the pad may vary depending on the pad composition and adhesion to the wound. In some cases, the force required to remove the pad may cause further damage to the wound, for example, by removing a scab that has formed. It is unclear how long an adhesive bandage should remain in place before replacing it with another or removing the adhesive bandage altogether.

Moreover, there is no specific guidance on pad construction and properties, how long the adhesive bandage should stay in place or the frequency of adhesive bandage replacement in order to reduce the time for wound healing.

Therefore, what is needed is an adhesive bandage comprising an absorbent pad with the ability to effectively absorb wound exudate while being readily removable without hindering the healing process. Moreover, a treatment method is required that specifies the duration of adhesive bandage application and replacement frequency to reduce the time for wound healing.

### SUMMARY OF THE INVENTION

We have found that an adhesive bandage comprising a hydrocolloid pad with specific absorbency and peel strength effectively removes wound exudate while enabling facile removal. The bandage includes a backing material comprising a sheet having an adhesive layer disposed thereon having a first color. The hydrocolloid pad has an absorbency (24 hr) of between about 2 and about 6 g/g and includes a hydrocolloid layer forming a first surface thereof and a polymeric film layer having a second color associated therewith different than the first color of the backing material forming a second surface thereof. The hydrocolloid pad is disposed in facing relation and adhered to the adhesive layer of backing material. The first surface having a dry peel strength of between about 0 and about 700 gf, a wet peel strength of between about 0 and 150 gf. The release liner system includes a first sheet superposed on translucent hydrocolloid pad; and second and third sheets superposed over adhesive layer of the backing material and the first sheet of the release liner system.

In a second aspect of the present invention, we have found that replacing the inventive adhesive bandage summarized in the preceding paragraph every 1-2 days leads to about 32% to about 71% better healing based on 7-day average of percentage of subjects showing improvement of general wound appearance compared to standard of care based on clinical testing results.

Further objects and advantages of the invention will be readily apparent to those skilled in the art from the following detailed description, taken in conjunction with the sheets of drawings.

### BRIEF DESCRIPTION OF THE DRAWINGS

Embodiments of this invention will now be described in greater detail, by way of illustration only, with reference to the accompanying drawings, in which:

FIG. 1 is a plan view of the skin contacting face of a novel adhesive bandage showing a backing material and absorbent hydrocolloid pad;

FIG. 2 is a side view of the novel adhesive bandage of FIG. 1; and

FIG. 3 is an exploded side view of the novel adhesive bandage of and a release liner system.

### DETAILED DESCRIPTION OF THE INVENTION

The present invention relates to novel adhesive bandages and methods of use shown clinically to improve the wound healing process.

As used herein the specification and the claims the term “colorfast” and variants thereof means that the pigment or dye providing the color associated with the polymeric film of the hydrocolloid pad does not migrate to the hydrocolloid layer of the pad or to any underlying wound surface.

As used herein the specification and the claims the terms “dry peel strength” and “wet peel strength” and variants thereof refer to 90° peel strength as defined below.

An adhesive bandage comprising a hydrocolloid pad with absorbency and peel strength effectively removes wound exudate while enabling facile removal without unnecessarily disturbing the wound or the healing process, even if the bandage is replaced every 1-2 days, which can lead to about 32% to about 71% better healing based on 7-day average of percentage of subjects showing improvement of general wound appearance compared to standard of care based on clinical testing results.

We have found that prior art hydrocolloid adhesive bandages generally have high adhesion to both the wound and surrounding skin. These prior art hydrocolloids are typically designed to stay on wound for several days or more. The adhesion will naturally decrease with absorption of large amounts of wound exudates. Removing hydrocolloids from a wound bed after only a day or 2, without hydrocolloids absorbing sufficient volume of exudates may result in disruption of the epithelial tissues. We have found that absorbent hydrocolloid pads designed with low adhesion allow for daily/every 48-hour replacement while helping to prevent re-injury of epithelial tissues during the bandage removal process.

In addition, we have found that effectively sealing the wound through an effective liquid barrier incorporated into the backing material of an adhesive bandage coupled with a low tack hydrocolloid pad also promotes better healing than a system that fails to effectively seal the wound from the environment.

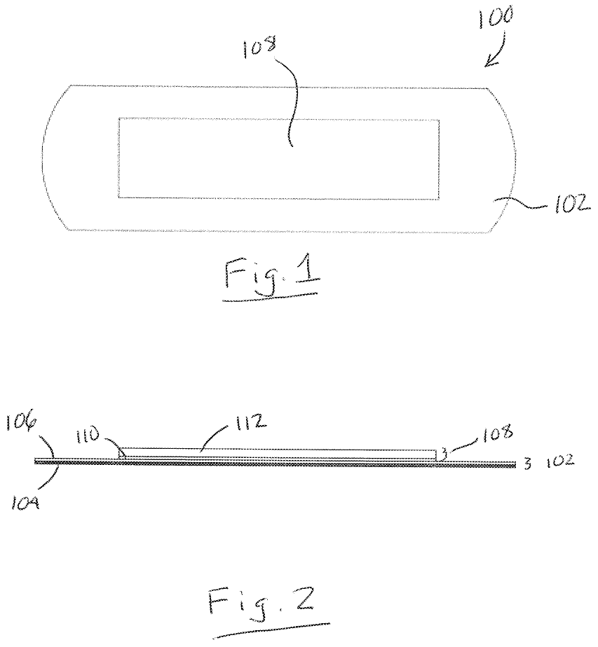

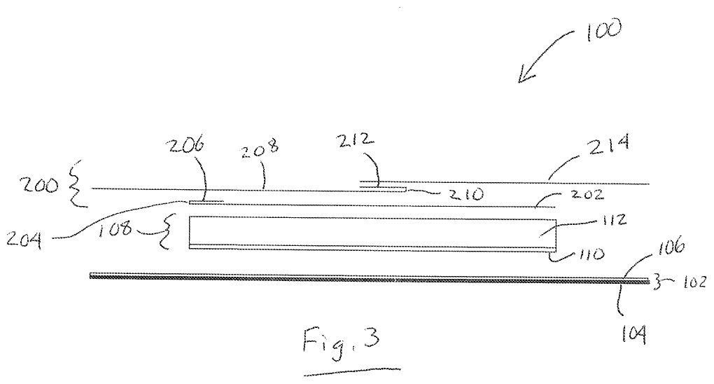

FIGS. 1-3 illustrate an embodiment of the novel adhesive bandage 100 of the present invention. The novel adhesive bandage 100 comprises a backing material 102 formed of a sheet 104 and having an adhesive 106 applied to the sheet 104 and a hydrocolloid pad 108 having a polymeric film 110 of lesser area than that of the backing material 102 and adhered thereto a hydrocolloid material 112 is disposed on and substantially coextensive the polymeric film 110.

A release liner system 200 protects the adhesive surfaces of the adhesive bandage and includes a first release liner 202 disposed on and substantially coextensive with the hydrocolloid material 112. The first release liner 202 has a fold 204 to provide a tab 206 to facilitate grasping the first release liner 202 to remove it from the hydrocolloid material 112. A second release liner 208 having a fold 210 forming a tab 212 and a third release liner 214 are superposed over the backing material 102 and hydrocolloid pad 108 and are substantially coextensive with the perimeter of the adhesive bandage 100.

The sheet 104 of backing material 102 may be comprised of woven fabric or polymeric films such as polyolefin (e.g., polyethylene) polyvinylchloride (PVC), polyester, latex or polyurethane films. A preferred backing material is a non-woven polyurethane laminated to a polyurethane film. Preferably the non-woven polyurethane and polyurethane film have weights of 40 and 30 grams per square meter (gsm), respectively. The sheet 104 may be formed in any shape including rectangles, squares, circles, ellipsoids or of symmetrical or asymmetrical design. The sheet 104 has a first color (at least on the adhesive-coated surface). Colors matching skin tones are preferred. Brown is a more preferred color for the backing material.

The adhesive 106 may be a tacky, biocompatible substance. Acrylic polymers are preferred adhesives. Preferably, the acrylic adhesive has a weight of 25 gsm when applied to the backing material. The adhesive may be applied as a coating to portions, or the entire surface area of the intended skin-facing side of a backing material.

The hydrocolloid pad 108 may be formed of a polymeric film 110 coated with a hydrocolloid material 108 having adhesive characteristics. The polymeric film may be comprised of polymers that are synthetically manufactured. The polymeric film is preferably a polyurethane film. The polymeric film may be advantageously pigmented so it is visible through the hydrocolloid, which is typically translucent, thereby providing the hydrocolloid pad with a second color that is discernible from the first color. As used in the specification and claims, a “discernible color” is one that is readily observed and distinguished by the human eye from its environment (i.e., the first color of the backing material). According to R. F. Witzel, R. W. Burnham, and J. W. Onley. “Threshold and suprathreshold perceptual color differences” J. Optical Society of America, Vol. 63, Issue 5, pp. 615-625 (1973), color can be precisely described in co-ordinate systems, such as L*a*b*. In the L*a*b* system can be plotted as a sphere that contains hundreds of thousands of different colors. In order to calculate the difference between two colors within the L*a*b* system, a trigonometric calculation of the distance between the colors' respective points in the sphere. The distance between two colors is known as the Delta-E and is an industry standard that is overseen by the International Commission on Illumination. The average human eye cannot detect any color difference with a Delta-E value of 3 or less. Thus, a discernible color difference will have a Delta-E value of greater than 3.

The second color of the hydrocolloid pad 108 facilitates alignment of the hydrocolloid pad of the adhesive bandage with the wound. The polymeric film 110 may be pigmented by printing on the surface of the polymeric film or by tinting such that the pigment is disposed throughout the entire film. A polymeric film 110 which is pigmented by printing on the surface of the film may maintain some translucency such that the color of the backing material to which the hydrocolloid pad is affixed would still contribute to the perceived color of the pad on the adhesive bandage. For example, a hydrocolloid pad 108 comprising a polymeric film 110 with a printed blue color on the surface and affixed to a brown backing material may appear gray or bluish gray. On the other hand, a polymeric film 110 which is pigmented by disposing the pigment throughout the film would be more substantially opaque. Thus, a hydrocolloid pad 108 comprising a polymeric film 110 with a blue pigment disposed throughout the film and affixed to a brown backing material would still appear predominantly blue. In either case, it is preferred that the pigmented polymeric film 110 be colorfast—i.e., the pigment will not migrate from the film under conditions of storage or use. Advantageously, such a colorfast pigment would not migrate to contact the skin or wound to any degree since it is on the side of the hydrocolloid not touching the wound. In addition, a pigment printed on the side of the polymeric film 110 facing away from the hydrocolloid layer and towards the backing material 102 would be well-isolated from the The hydrocolloid material 112 may be one or more of those recited in US2022/0111111 as follows. A hydrocolloid may be a water absorbing and/or water-swellable material. The hydrocolloid may be synthetic or naturally occurring. The hydrocolloid may, for example, be crosslinked. The hydrocolloid may, for example, be selected from polyvinyl alcohol, polyvinyl acetate, polyvinylpyrolidone, polyacrylic acid, polyhydroxyalkyl acrylates, polyacrylamides, high molecular weight polyethylene glycols, polypropylene glycols, carboxymethylcellulose (CMC) (e.g. sodium carboxymethyl cellulose, cross-linked sodium carboxymethylcellulose, and/or crystalline carboxymethylcellulose), pectin, gelatin, high molecular weight carbowax, carboxypolymethylene, carboxymethyl starches, alginates, carrageenan, gelatin, citrus pectin, powdered pectin, synthetic or natural gums such as guar gum, gum Arabic, locust bean gum, tragacanth gum, dextran, starches, karaya, starch-acrylonitrile graft copolymer and mixtures thereof. Preferred hydrocolloids may be carboxymethylcellulose, alginates, and pectin. The hydrocolloid may be particles, for example having an average particle size of from about 1 μm to about 400 μm.

Preferred hydrocolloid pads 108 have a thickness of between about 0.1 and about 0.6 mm. More preferred hydrocolloid pads have a thickness of 0.2 to 0.46 mm. Preferred hydrocolloid pads have fluid absorbency (measured over a 24-hour period at ambient laboratory conditions) of between about 2 and about 6 grams of fluid absorbed per gram of hydrocolloid pad or 2-6 g/g. Additionally, the hydrocolloid pads preferably should have dry peel and wet peel strengths of between about 0 and about 700 grams-force (gf) and between about 0 and 150 gf, respectively. Advantageously, the wet peel strength is lower than the dry peel strength for a given hydrocolloid pad so that a corresponding adhesive bandage is easier and less painful to remove after it absorbs wound exudate. A dry peel to wet peel strength ratio of 3 to 9 is preferred. Dry peel adhesion is tested in accordance with ASTM D3330 (90° peel). Wet peel adhesion is tested as outlined, below (modified from dry peel adhesion, ASTM D3330 (90° peel)).

### Wet Adhesion (Wet Peel) 90° Peel Test Method:

### 1. Wet Adhesion Sample Preparation

1.1. Cut hydrocolloid sample to 0.75″×2″.
1.2. Lift the facing tab of the hydrocolloid 1 inch to where the adhesive side of the hydrocolloid is exposed, facing up.
1.3. Using a micropipette, apply 15 μL of 0.9% saline solution to the center of the exposed 0.75″×1″ adhesive side of the hydrocolloid.
1.4. Immediately after applying the saline, using a micro slide, cover the exposed 0.75″×1″ adhesive side with the drop on it, to where the drop will fully immerse the 0.75″×1″ portion of the adhesive side of the hydrocolloid.
1.5. Record the time and allow the microslide to stick to the hydrocolloid for 1 hour.
1.6. Once the hour has passed, you may now peel the glass from the hydrocolloid to proceed with the Peel Adhesion 90° testing protocol in accordance with ASTM D3330.

### 2. 90° Peel Sample Preparation

2.1. Visually examine the steel plate. The testing side is the side that has no identification label. If there are any scratches on the testing side, use a new plate.
2.2. Clean the steel plate with acetone and lint-free gauze/paper wipes. Steel plate shall be allowed to dry. Visually check to make sure there is no acetone residue left before starting next step.
2.3. To eliminate any potential elasticity during the peeling test, strong tape, such as GORILLA brand adhesive tape should be used as support for the specimen to be tested. Cut 2 inches of the adhesive tape and place it on the top (non-adhesive) side of the hydrocolloid test material. Cut any exposed adhesive side of the tape that is not being covered by the bandage.
2.4. Remove one of the release tabs to expose hydrocolloid test material.
2.5. Cover any exposed adhesive tape from step 2.3. Place the hydrocolloid test material (adhesive side down) on the steel plate.
2.6. Set the speed of the IMASS PR-1000-24 POWEROLL automatic roller test machine at 10±0.5 mm/s [24 in/min]. Place the steel plate at a suitable position so that the centerline of the hydrocolloid test sample (along lengthwise direction) overlaps with the centerline of the roller. The attached section will be rolled over twice in lengthwise direction. Prevent entrapment of air between the adhesive and the panel. Should this occur, discard the specimen.

### 3. 90° Peel Sample Procedure:

3.1. The specimen must be tested within 1 minute of sample preparation.
3.2. Confirm that Instron (adhesion tester) Series 5543 Model has been calibrated. Zero the force channel.
3.3. Set-up the tensile tester with a grip separation rate of 5 mm/s [12 in/min] and set the separation value at 50.8 mm [2.0 in] and a maximum crosshead travel at 76.2 mm [3.0 in].
3.4. Insert the exposed bottom grip of the sled apparatus into the bottom grip of the Adhesion tester. Make sure the sled is leveled (by using level or visually checking if the sled is tilted to one end). Make sure the sled travels freely.
3.5. Take the free end of the bandage and pull it up toward the top grip of the Adhesion Tester and secure bandage.
3.6. Start the test.
3.7. Remove the specimen after test is complete.
3.8. Record the results. Again, the hydrocolloid pads 108 designed with low adhesion allow for daily/every 48-hour replacement while helping to prevent re-injury of epithelial tissues during the bandage removal process.

The release liner system 200 includes a plurality of protective sheets that isolate the adhesive surfaces of the adhesive bandage before use. A first release liner 202 can be applied to the hydrocolloid material 112 of the hydrocolloid pad 108 to protect the tacky hydrocolloid surface before application of the adhesive bandage by the user. Suitable liner materials include, but are not limited to bleached Kraft paper, silicone coated on one side at least where contact with the hydrocolloid is made. A preferred release liner material is a bleached 50 gsm monolucid paper coated on one side with 20 gsm polyethylene and a silicone release formulation and having a glossy appearance on the coated side, optionally printed with removal instructions. The first release liner can be of the same dimensions as the hydrocolloid pad or can be of different dimensions to facilitate removal of the liner from the hydrocolloid pad. Where the liner is of different dimensions as the hydrocolloid pad, the liner can be larger in any one or all planar dimensions than the hydrocolloid pad. Further, the liner can have lines of weakness, such as scores, perforations or a folded extension that can serve as a pull tab to facilitate removal of the liner from the hydrocolloid pad.

Second and third release liners 208 and 214 may be of similar composition as that of the first release liner 202. The second and third release liners may be superposed over the adhesive layer 104 of the backing material 102 and the first release liner 202. The second and third release liners preferably completely cover the adhesive layer 104 on each side of the hydrocolloid pad and extend over at least a portion of a first release liner 202. More preferably, the second and third release liners have extensions (folded or unfolded) that can serve as pull tabs to facilitate removal. Taken as a whole, a first, second and third release liners function as a release liner system which facilitates use of the adhesive bandage in a manner that minimizes inadvertent microbial contamination of the hydrocolloid pad.

The adhesive bandages are individually wrapped in a wrapper (free of natural rubber latex), filled into cartons, and packed out into shipping containers. The wrapper is preferably made of white sterilizable Kraft paper—34 gsm with cohesive coating and optionally printed. A plurality of individually wrapped adhesive bandages are generally supplied in a box or carton containing about 2 to about 100 individually-wrapped adhesive bandages. The products are then sterilized in their final packaging using Electron beam (E-beam) radiation.

An advanced method of treating a wound in a skin surface using the novel adhesive bandages has also been developed. This advanced method comprises the steps of:
(a) removing the second and third sheets of the release liner system from the adhesive bandage described above;
(b) removing the first sheet of the release liner system from the hydrocolloid pad to expose the hydrocolloid pad having the second color associated therewith;
(c) aligning the second color associated with polymeric film of the hydrocolloid pad with the wound;
(d) adhering the adhesive bandage to the skin surface with the hydrocolloid pad superposed over the wound;
(e) maintaining the adhesive bandage on the skin surface for a treatment period of between 1 and 2 days; and
(f) repeating steps (a)-(e) for at least one additional treatment period of between 1 and 2 days.

Preferably, the novel adhesive bandage will be replaced every day for a least a period of 7 days.

The present invention will be better understood from a consideration of the following illustrative examples.

### EXAMPLES

The example that follows is based on a single center, randomized, comparator-controlled clinical trial which compared healing rates and infection protection for different treatment regimens including a standard of care adhesive bandage and a hydrocolloid pad. A total of 34 subjects completed study participation, with all 34 subjects included in the intent-to-treat (ITT) population. The subjects enrolled in this trial were 25- to 55-year-old males and females of Fitzpatrick skin types I-III who had uniform skin color on both volar forearms, and who had consented to participate in this clinical trial. At Screening (Visit 1; 3 to 7 days prior to Baseline), subjects were provided with an auxiliary cleanser to use on their forearms and for all body cleansing in place of their regular body cleanser for the duration of the study. At Baseline or Day 0 (Visit 2), a Sciton Er:YAG 2940 laser was used to induce partial-thickness (i.e. minor) wounds on the subjects' forearms. The wounds created by this method heal by the migration of epidermal cells from the dermal appendages located in the wound's base (dermal islands) and/or wound borders, and mimic minor wounds similar to real life scraped skin, typically healing in less than 16 days if left untreated. Each wound test site for each subject was randomly assigned to one of the following:

| Example | Treatment description |
|---|---|
| Comparative Example A | Uncovered wound observed through 7 days. |
| Comparative Example B | Standard of care adhesive bandage |
| | applied initially on Day 0 and |
| Example 1 | on each of 6 days thereafter. |
| | Hydrocolloid pad applied initially |
| | on Day 0 and on each of 6 |
| | days thereafter. |

The treatment components used in each of the examples are further described below:

### Standard Adhesive Bandage

The standard of care bandage comprised a flexible fabric material backing material and an absorbent pad comprised of an absorbent nonwoven material consisting of polyester fibers and high-density polyethylene (HDPE) netting film, together as one structure, and embossed with a hexagonal pattern design (Band-Aid® Brand Adhesives Bandages, Flexible Fabric, All One Size, UPC Code 381370044314).

### Hydrocolloid Pad

The hydrocolloid pad (H020 AWC Soft-Pro® Hydrocolloid) was supplied by Scapa Healthcare (Orangeburg, NY). The pad comprised a polyurethane film, hydrocolloid (containing synthetic elastomeric polymers, tackifier resin, plasticizer and hydrocolloid particles) and siliconized paper (paper removed before application to the wound).

### Wound Assessment

Each wound site was photographed (through Day 7) and assessed at specified intervals by clinical grading of wound healing parameters. General Wound Appearance was assessed clinically using the following scoring system:

| Appearance Assessment | Score |
|---|---|
| Poor | 0 |
| Fair | 1 |
| Good | 2 |
| Very Good | 3 |
| Excellent | 4 |

The following table summarizes the mean General Wound Appearance scores for the 34 study participants for each treatment from Day 0 through Day 7:

TABLE 1
Mean General Wound Appearance (n = 34)

| | | Comparative | | |
|---|---|---|---|---|
| | Comparative | Example B | Example 1 | Percentage of |
| | Example A | Adhesive | Hydrocolloid | Subjects Showing |
| | Untreated | Bandage | Bandage | Improvement* |
| Day 0 | 0 | 0 | 0 | N/A |
| Day 1 | 0.35 | 0.59 | 0.75 | 32 |
| Day 2 | 0.53 | 0.76 | 1.18 | 59 |
| Day 3 | 0.63 | 0.85 | 1.19 | 56 |
| Day 4 | 0.80 | 1.00 | 1.32 | 64 |
| Day 5 | 1.24 | 1.32 | 1.71 | 71 |
| Day 6 | 1.63 | 1.69 | 2.09 | 71 |
| Day 7 | 1.96 | 2.04 | 2.50 | 71 |

*Percentage of subjects showing improvement in General Wound Appearance Score when the individual subject results with the hydrocolloid bandage of Example 1 are compared to those obtained with the standard adhesive bandage of Comparative Example B.

Taken as a whole, these data demonstrate that the daily application of a fresh hydrocolloid pad results in a significantly faster healing rate than the daily application of a standard of care adhesive bandage or no treatment at all. The percentage of subjects showing improvements in General Wound Appearance Score when using the hydrocolloid bandage of Example 1 progressively increased from 32% after 1 day to 71% after 5-7 days. The 7-day average of the percentage of subjects showing improved General Wound Appearance compared to standard of care was 60%.

As part of the same clinical study, a Composite Healing Score was determined which was calculated from the clinical grading of general wound appearance as discussed above as well as other wound healing parameters as follows: Composite Healing Score= [general wound appearance score+smoothness score+epithelial confluence score]-[erythema score+edema score+crusting/scabbing score].

The Composite Healing Score, on a 25-point scale (−12 thru+12), is indicative of the extent of wound healing and was calculated for each wound site at each evaluation day. The greater the composite healing score, the great the extent of wound healing. The other wound healing parameters were clinically assessed as follows:

### Clinical Grading of Wound Healing-Smoothness

0=rough, uneven wound
1=mild smoothness
2=moderate smoothness
3=extensive smoothness
4=complete smooth, even wound

### Clinical Grading of Wound Healing-Epithelial Confluence

0=none, no epithelial coverage
1=slight (up to 30%)
2=moderate (31-60%)
3=extensive (61-90%)
4=almost complete or complete (91-100%), covered with a full layer of new epithelial growth

### Clinical Grading of Wound Healing-Erythema

0=none/absent
1=mild
2=moderate
3=marked
4=severe

### Clinical Grading of Wound Healing-Edema

0=none/absent
1=mild
2=moderate
3=marked
4=severe

### Clinical Grading of Wound Healing-Crusting/Scabbing

0=none
1=slight (up to 30%)
2=moderate (31%-60%)
3=extensive (61%-90%)
4=almost complete or complete (91%-100%)

The following table summarizes the mean Composite Healing Scores for the 34 study participants for each treatment from Day 0 through Day 7:

TABLE 2
Mean Composite Healing Score (n = 34)

| | | Comparative | | |
|---|---|---|---|---|
| | Comparative | Example B | Example 1 | Percentage of |
| | Example A | Adhesive | Hydrocolloid | Subjects Showing |
| | Untreated | Bandage | Bandage | Improvement* |
| Day 0 | 12 | 12 | 12 | N/A |
| Day 1 | −3.51 | −2.10 | −0.60 | 82 |
| Day 2 | −3.93 | −3.03 | −0.40 | 91 |
| Day 3 | −4.03 | −2.91 | −1.09 | 88 |
| Day 4 | −4.05 | −3.23 | −0.88 | 82 |
| Day 5 | −3.01 | −2.26 | 0.15 | 79 |
| Day 6 | −1.71 | −1.15 | 1.63 | 85 |
| Day 7 | −0.31 | −0.01 | 3.04 | 85 |

*Percentage of subjects showing improvement in Composite Healing Score when the individual subject results with the hydrocolloid bandage of Example 1 are compared to those obtained with the standard adhesive bandage of Comparative Example B.

The Composite Healing Score data also demonstrate that the daily application of a fresh hydrocolloid pad results in a significantly faster healing rate than the daily application of a standard of care adhesive bandage or no treatment at all. The percentage of subjects showing improvements in Composite Healing Score when using the hydrocolloid bandage of Example 1 was consistently between 79 and 91% on Days 1-7. The 7-day average of the percentage of subjects showing improved Composite Healing Scores compared to standard of care was 85%.

### Example 2—Ink Migration Test

The purpose of this test was to confirm whether a hydrocolloid pad comprising a blue-printed polyurethane film-hydrocolloid laminate is colorfast. Hydrocolloid pad roll stock (1 inch in width) was prepared by adhering a hydrocolloid (product code H80 from Scapa) material to the blue side of a polyurethane film, which was previously printed on one surface with a blue ink. The thickness of the hydrocolloid pad was 0.45 mm. A test disc was cut from the hydrocolloid pad and placed in a jacketed Franz Diffusion Cell (ELABE40) with hydrocolloid side of the pad facing the diffusion medium (0.9% saline) and clamped tightly into place to prevent any leakage. The temperature of the diffusion medium was maintained at 37° C., and the duration of the exposure was 72 hours. After this period, the resulting solution was analyzed on a UV-visible spectrophotometer to check for the presence of any peaks ascribable to the blue color. No peaks were observed in the visible range (approximately 380 to 750 nm) including the 450-480 nm range, which would correspond to the blue color. The above protocol was completed three additional times with three new samples, and each time there was no evidence of any peaks. These data confirm that this hydrocolloid pad is colorfast under the testing conditions and further suggest that there would be no color migration when contacting wounds on human skin.

What is claimed is:

1. An adhesive bandage comprising:
(a) a backing material comprising a sheet having an adhesive layer disposed thereon having a first color;
(b) a hydrocolloid pad having an absorbency (24 hr) of between about 2 and about 6 g/g, the pad comprising a hydrocolloid layer forming a first surface thereof and a polymeric film layer having a second color associated therewith different than the first color of the backing material forming a second surface thereof disposed in facing relation and adhered to the adhesive layer of backing material, the first surface having a dry peel strength of between about 0 and about 700 gf, a wet peel strength of between about 0 and 150 gf; and
(c) a release liner system comprising:
(i) first sheet superposed on translucent hydrocolloid pad; and
(ii) second and third sheets superposed over adhesive layer of the backing material and the first sheet of the release liner system.

2. The adhesive bandage of claim 1 wherein the backing material comprises a liquid barrier.

3. The adhesive bandage of claim 2 wherein the sheet comprises a laminate of a nonwoven fabric and a polymeric film.

4. The adhesive bandage of claim 3 wherein the polymeric film of the backing material comprises polyurethane.

5. The adhesive bandage of claim 1 wherein the adhesive of the backing material comprises a polyacrylic adhesive.

6. The adhesive bandage of claim 1 wherein the hydrocolloid pad is translucent.

7. The adhesive bandage of claim 1 wherein the hydrocolloid pad has a thickness of about 0.1 to 0.6 mm.

8. The adhesive bandage of claim 1 wherein the polymeric film of the hydrocolloid pad comprises polyurethane.

9. The adhesive bandage of claim 1 wherein the second color associated with the polymeric film of the hydrocolloid pad is printed on a surface of the polymeric film.

10. The adhesive bandage of claim 1 wherein the second color associated with the polymeric film of the hydrocolloid pad is disposed in the polymeric film.

11. The adhesive bandage of claim 1 wherein the second color associated with the polymeric film is colorfast.

12. The adhesive bandage of claim 1 wherein the first, second, and third sheets of the release liner system are silicone-coated polyethylene sheets.

13. The adhesive bandage of claim 1 wherein the first sheet of the release liner system has a fold disposed at one end of the hydrocolloid pad and returns partially toward an opposite end of the hydrocolloid pad to form a greppable tab to aid in removal thereof.

14. A method of treating a wound in a skin surface comprising the steps of:
(a) removing the second and third sheets of the release liner system from the adhesive bandage of claim 1;
(b) removing the first sheet of the release liner system from the hydrocolloid pad to expose the hydrocolloid pad having the second color associated therewith;
(c) aligning the second color associated with polymeric film of the hydrocolloid pad with the wound;
(d) adhering the adhesive bandage to the skin surface with the hydrocolloid pad superposed over the wound;
(e) maintaining the adhesive bandage on the skin surface for a treatment period of between 1 and 2 days; and
(f) repeating steps (a)-(e) for at least one additional treatment period of between 1 and 2 days.

15. The method of claim 14 wherein the step of adhering the adhesive bandage to the skin further comprises sealing the backing material to the skin surface around the wound to isolate the wound from its environment.
